(19) United States
(12) Patent Application Publication
(10) Pub. No.: US 2018/0028275 A1
(43) Pub. Date: Feb. 1, 2018

(54) Dental Implant Identification System

(71) Applicant: Universal Implant Technologies Pty Limited, Menai, NSW (AU)

(72) Inventors: Peter Bradley, Menai, NSW (AU); Magnus Karlsson, Kungsnägen (SE); Anthony Chrisis, Menai, NSW (AU); Stan Yannopoulos, Menai, NSW (AU); Michael Counsel, Menai, NSW (AU)

(21) Appl. No.: 15/552,803

(22) PCT Filed: Feb. 23, 2016
(86) PCT No.: PCT/AU2016/000055

§ 371 Date: Aug. 23, 2017

(30) Foreign Application Priority Data
Feb. 23, 2015 (AU) 2015900637

(51) Int. Cl. A61B 90/98; G06K 19/077; G06K 7/10; A61C 8/00

(52) U.S. Cl. CPC A61B 90/98; A61C 8/0022; A61C 8/0089; G06K 19/07773; G06K 7/10316; A61C 2204/005

(57) ABSTRACT

A dental implant identification system of the non-contact type has a dental implant (260) and a non-contact tip (262) of a reader probe (263). The dental implant (260) has a non-contact RFID tag (264), which is cylindrical in shape, positioned immovably inside an open cylindrical cavity (266) within the main body of the dental implant (260), The non-contact tip (262) of the reader probe (263) includes, at its leading end, a reader antenna coil (268) (which is a transmitter receiver element in the form of a power coil), a coil positioning spring (270) and a reader positioning collar (272). The reader antenna coil (268) is electrically connected to, and receives its power through, wiring (273) from a match circuit (274) along which current flows. In use, the contact tip (262) of the powered on reader probe (263) is inserted through the opening of the cavity (266) of the dental implant (260) until further insertion is prevented by the reader positioning collar (272) becoming wedged ill the opening, and the tag antenna coil (276) and the reader antenna coil (268) are magnetic field coupled. The non-contact RFID tag (264) is passively powered by electromagnetic wave transmissions from the reader antenna coil (268) of the reader probe and received by the tag antenna coil (276).

### TECHNICAL FIELD

The present invention relates to dental implants and, in particular, to devices, systems and methods which allow radio frequency identification of dental implants. More particularly, the present invention relates to radio frequency identification systems of both the non-contact type and of the contact type which can be used for identifying dental implants

### BACKGROUND ART

Dental implants are root replacement devices used in dentistry to provide a support for prosthetic teeth or other dental appliances. They are screwed into a suitably prepared site in the jaw bone with the screw serving as a fixture onto which a prosthetic tooth or other dental appliance may be mounted. Dental implants have been h use for over 40 years and have been extremely successful in treating patients with tooth loss. The efficacy and success of dental implants and the rising demand for cosmetic dentistry worldwide across all age groups has led to an exponential growth in the industry and to a very large number of different types or brands at dental implants that have been made available.

This growth has resulted hi a vast array of design variations in dental implants with incompatibility existing between dental implants from different manufacturers. In general, the industry is characterized by a lack of standardization in the devices and systems that are used at the clinical level, and this incompatibility at the prosthetic interface is compounded by the large range of possible dental implants. This poses an extreme challenge for the clinician when faced with re-servicing existing dental implants, such as is required when replacing a broken prosthetic tooth. Frequently, the availability of proper dental records is lacking, especially when patients move locations, and so it may be impossible in those circumstances to reliably identify the nature of the existing dental implant in need of re-servicing.

The current method of identification relies largely on radiographic imaging. However, identification of the brand of a dental implant screwed in the jaw bone using radiographic images of brand specific features is difficult and unreliable and requires the clinician to stay abreast of a myriad of dental implant systems that are regularly changing.

Radio frequency identification (RFID) systems are well known in some other industries for their usefulness in quickly and reliably identifying small objects. RFID systems conventionally include an RFID tag which may be programmed and interrogated by a reader device. The RFID tag has an integrated circuit with a radio transceiver and antenna. The integrated circuit may be programmed by the reader device to contain identification and other application specific information, RFID systems compared to other identification means, such as physical identifiers, offer the significant advantage of storing a large amount of digital information in a physically small form.

US Patent Application Publication No. 2009/0155744 A1 (by Jandali) discloses a dental implant identification system based on the concept of using radio frequency identification (RFID) devices within dental implant screws as a means of identification. Other than disclosing that an RFID tag is positioned at the bottom of an internal bore within the dental implant screw, no information is provided in Jandali as to the necessary technical details of the RFID tag and its antenna. In particular, there is no disclosure of the structure (or configuration) and orientation of the RFID and its antenna within the screw.

Currently, there are no known dental implants that are being manufactured with RFID tags. The common structure of RFID tags in the time leading up to Jandali consisted of a tag mounted on a carrier substrate with the antenna printed on a earlier board. This is inexpensive to construct and the flat planar configuration is suitable for many applications. However, the magnetic field coupling from a reader antenna to the tag antenna and the resulting received voltage supplying the tag will be extremely weak owing to the small flat planar configuration of the tag antenna. The received voltage (or received signal strength) will be too weak to power up the circuitry within the tag which requires a minimum of 1 volt in most tags. Furthermore, tag antennae having a flat planar configuration present difficulties in the manner in which they may be located within a confined space, such as within the cylindrical bore of a dental implant screw.

The use of RFID tags when placed in a very small, completely enclosed, metal cavity is extremely challenging. Placing an RFID tag within a very small space at the bottom of a cavity within a dental implant screw with a volume typically of <5 mm³ poses severe performance limitations. These performance limitations arise from the small size of the antenna and the effect of the surrounding metal on the performance of the radio transceiver and antenna.

Dental implants are typically constructed of titanium or zirconium and associated alloys since these materials provide the necessary strength and biocompatibility requirements for prolonged and effective use. These materials, and any other material of similar conductivity and thickness, provide a shield that significantly attenuates the radio frequency signal. The amount of attenuation is an exponential function of the skin depth of the material(s) used to construct the dental implant.

Further deterioration of the received signal strength occurs due to the close proximity of the metal to the tag antenna Which decreases the resonant signal by degrading the antenna's electrical properties. The often deleterious effect of materials (especially conductive materials) in close proximity to antennas is well known. Due to these extremely challenging conditions, RFID devices have not yet been used for the purpose of identification of dental implants.

The present inventors have recognised that RFID systems offer the potential to address all the problems associated with current methods of identifying dental implants and have also recognised that the vast information storage capability of RFID systems may be used for patient dental record and other user information as desired by the clinician.

The present inventors have also recognised that any RFID systems to be used for identifying dental implants can be either of the non-contact type or of the contact type.

However, the present inventors have equally recognised that for REID systems to perform at a sufficiently high level when used for identifying dental implants by way of RFID tags positioned therewithin and for storing information, significant changes to the configuration (i.e. structure and/or orientation) of at least the REID tag are needed.

It is a first discovery of the present inventors that, for a non-contact type of RFID system useful for identifying dental implants, a coil structure of the RFID tag antenna positioned within a dental implant is more advantageous than a flat planar structure, and that such an RFID tag antenna coil must be orientated in a particular manner within a dental implant to optimize the magnetic field coupling from a reader antenna to the tag antenna and hence optimise the received signal strength needed to power up the circuitry within the tag.

It has been found by the present inventors that such a non-contact type of RFID system useful for identifying dental implants may utilize a reader antenna that can either be positioned outside the dental implant or inside a tag containing cavity of the dental implant.

It is a second discovery of the present inventors that, for a contact type of RFID system useful for identifying dental implants, an RFID tag in the form of an integrated circuit (IC) between two contact electrodes and positioned inside a cavity of a dental implant can be used, and that such an RFID tag circuit may be activated by contact with a tip of a reader contact probe inserted within the cavity of the dental implant.

It has been found by the present inventors that, by putting these discoveries into practical implementation in an RFID system for identifying dental implants, the aforementioned problems and shortcomings of the prior art can be overcome or least substantially ameliorated,

### SUMMARY OF INVENTION

According to one aspect of a first form of the present invention, there is provided a dental implant identification system of the non-contact type, comprising:
(a) a dental implant having a radio frequency identification tag which stores information for identification of the dental implant, the tau including a tag antenna coil having a first coil axis,
(b) a reader device for activating the tag and for reading the information stored on the tag when the information is transmitted from the tag upon activation thereof, the reader device including a reader antenna coil having a second coil axis and
(c) information processing means for processing the identification information read by the reader device,

wherein the radio frequency identification tag is so configured within the dental implant to provide an optimal reading of the identification information when the reader antenna coil is positioned alongside the tag antenna coil and the second coil axis is substantially aligned with the first coil axis, and when the reader device activates the tag.

Preferably, the radio frequency identification tag is located inside a cavity within main body of the dental implant.

In one preferred arrangement, the reader device is configured such that the reader antenna coil, when positioned alongside the tang antenna coil and when the first coil axis and the second coil axis are substantially aligned, is outside the main body of the dental implant.

In another preferred arrangement, the reader device is configured such that the reader antenna coil, when positioned alongside the tag antenna coil and when the first coil axis and the second coil axis are substantially aligned, is inside the cavity of the main body of the dental implant.

Preferably, the information processing means is a computer associated with the reader device for accessing a database containing information relating to the identification information.

It is preferred that the radio frequency identification tag further includes an information storage element and an integrated control circuit electrically connected between the tag antenna coil and the storage element and operable to activate the storage element in response to a voltage being generated in the tag antenna coil when the tag antenna coil and the reader antenna coil are magnetic field coupled.

In a preferred form, the identification information is stored on a microchip.

The tag antenna coil preferably operates as a passive power supply for the tag.

In one preferred embodiment, the reader device has single reader antenna coil.

In another preferred embodiment, the reader device has two reader antenna coils.

According to another aspect of the first form of the present invention, there is provided a dental implant for engagement to a jaw bone, the dental implant comprising a main body having a longitudinal axis and an outer threaded surface for screwing the main body into the jaw bone in the direction of the longitudinal axis, the main body having an abutment for connecting a prosthetic tooth thereto, a cavity within the main body, a radio frequency identification tag positioned within the cavity for storing information for identification of the implant, the tag including an integrated circuit and a tag antenna coil having a tag coil axis that is orientated perpendicularly to the longitudinal axis of the main body, whereby the tag antenna coil of the radio frequency identification tag is able to be magnetic field coupled with a reader antenna coil of a reader device when the reader antenna coil is positioned alongside the tag antenna coil and when a reader coil axis of the reader antenna coil is orientated substantially perpendicularly to the longitudinal axis of the main body, the magnetic field coupling resulting in a voltage being generated in the tag antenna coil that is of a signal strength sufficient to cause the integrated circuit within the tag to be powered up so that the information can be read by the reader device.

According to tine aspect of a second form of the present invention, there is provided a dental implant identification system of the contact type, comprising:
(a) a dental implant having a main body, a cavity within the main body, and a radio frequency identification tag located inside the cavity, the radio frequency identification tag storing information for identification of the dental implant and including an integrated circuit between two contact electrodes,
(b) a reader device for activating the tag and for reading the information stored on the tag When the information is transmitted from the tag upon activation thereof, the reader device including a reader contact terminal, and
(c) information processing means for processing the identification information read by the reader device,

wherein the radio frequency identification tag is so configured within the dental implant to provide an optimal reading of the identification information when the reader contact terminal is inserted into the cavity and contacted against one of the contact electrodes, and when the reader device activates the tag.

Preferably, the information processing means is a computer associated with the reader device for accessing a database containing information relating to the identification information.

In a preferred form, the identification information is stored on a microchip.

According to another aspect of the second form of the present invention there is provided a dental implant for engagement to a jaw bone, the dental implant comprising a main body having a longitudinal axis and an outer threaded surface for screwing the main body into the jaw bone in the direction of the longitudinal axis, the main body having an abutment for connecting a prosthetic tooth thereto, a cavity within the main body, a radio frequency identification tag positioned within the cavity for storing information for identification of the implant, the tag including an integrated circuit between two contact electrodes, whereby one of the contact electrodes of the radio frequency identification tag is able to be contacted by a reader contact terminal of a reader device when the reader contact terminal is inserted within the cavity, the contact resulting in a voltage being generated in the integrated circuit that is of a signal strength sufficient to power up the integrated circuit so that the information can be read by the reader device.

Preferably, the radio frequency identification tag includes a printed circuit board on which is mounted an integrated circuit between a first contact electrode and a second contact electrode.

It is preferred that the printed circuit board and the integrated circuit are covered by a protective moulding that has a high electrical resistance.

In a preferred form, the first contact electrode presents a contact surface, such as in the shape of a conical depression, facing towards an opening of the cavity, and the second contact electrode presents a contact surface, such as in the shape of a conical projection, facing towards, and abutting, a base (or closed end) of the dental implant.

In a particularly preferred embodiment, when a contact tip of a powered on reader device is inserted through the opening of the cavity of the dental implant and the reader contact terminal of the reader device is pressed against, or contacts, the first contact electrode of the radio frequency identification tag, and a reader positioning collar of the reader device is wedged against, or contacts, the opening of the cavity in the main body, the interconnection of the aforementioned components closes an electrical circuit along which current flows between the reader device and the radio frequency identification tag of the dental implant.

There has been thus outlined, rather broadly, the more important features of the invention in order that the detailed description thereof that follows may be better understood and put into practical effect, and in order that the present contribution to the art may be better appreciated.

There are additional features of the invention that will be described hereinafter. As such, those skilled in the art will appreciate that the conception, upon which the disclosure is based, may be readily utilized as the basis for designing other devices, systems and methods for carrying out the objects of the present invention. It is important, therefore, that the broad outline of the invention described above be regarded as including such equivalent constructions in so far as they do not depart from the spirit and scope of the present invention.

### BRIEF DESCRIPTION OF THE DRAWINGS

The invention will be better understood and objects other than those set forth above will become apparent when consideration is given to the following detailed description thereof. Such description makes reference to the accompanying drawings, in which:

FIG. 1 is a schematic front sectional view of a dental implant identification system of the non-contact type showing only a dental implant and a single reader antenna coil, the configuration being according to a first embodiment of the invention,

FIG. 2 is a perspective view of a reader device containing a single reader antenna coil that can be used with the dental implant of the system shown in FIG. 1,

FIG. 3 is a side view of the reader device shown in FIG. 2,

FIG. 4 is a front sectional view through A-A of the reader device shown in FIG. 3,

FIG. 5 is a top view of the reader device shown in FIG. 2,

FIG. 6 is a side sectional view through D-D of the reader device shown in FIG. 5,

FIG. 7 is a schematic front sectional view of a dental implant identification system of the non-contact type showing only a dental implant and two reader antenna coils at respective opposed sides of the dental implant, the two reader antenna coils being positioned so as to have their axes aligned with the axis of a tag antenna coil positioned inside the dental implant, the configuration of all of these components being according to a second embodiment of the invention,

FIG. 8 is a perspective view of a reader device containing two reader antenna coils that can be used with the dental implant of the system shown in FIG. 7,

FIG. 9 is a side view of the reader device shown in FIG. 8,

FIG. 10 is a front sectional view through A-A of the reader device shown in FIG. 9,

FIG. 11 is a top view of the reader device shown in FIG. 8,

FIG. 12 is a side sectional view through A-A of the reader device shown in FIG. 11

FIG. 13 is a block and circuit diagram showing the key electronic components and circuitry design of a reader device that can be used in any of the first and second embodiments of the invention, and showing its interaction with the radio frequency identification tag of the dental implant and with We information processing means,

FIG. 14 is an alternate block and circuit diagram similar to that of FIG. 13,

FIG. 15 is a schematic front sectional view of a dental implant identification system of the non-contact type showing only a tag antenna coil of a dental implant and two reader antenna coils at respective opposed sides of the tag antenna coil, the two reader antenna coils being positioned so as to have their axes offset from the axis of the tag antenna coil positioned inside the dental implant, the configuration of all of these components being according to a third embodiment of the invention,

FIG. 16 is a graph showing (magnetic field induced voltage) signal strength received on the tag antenna coil versus degree of alignment of axes of two reader coils at respective opposed sides of the dental implant of the system shown in FIG. 15,

FIG. 17 is a schematic top sectional view of a dental implant identification system showing only a dental implant and twelve reader antenna coils at respective evenly spaced apart locations radially around the side of the dental implant, the twelve reader antenna coils being positioned so that at least two of those coils have their axes aligned substantially with the axis of the tag antenna coil positioned inside the dental implant, the configuration of all of these components being according to a fourth embodiment of the invention,

FIG. 18 is a schematic top sectional view of a dental implant identification system showing only a dental implant and six reader antenna coils divided into two groups of three coils at respective opposed sides of the dental implant, the six reader antenna coils being positioned so that at least two of those coils have their axes aligned substantially with the axis of the tan antenna coil positioned inside the dental implant, the configuration of all of those components being according to a fifth embodiment of the invention,

FIGS. 19A, 19B, 19C and 19D are schematic views of a double sided circuit board for use in the radio frequency identification tag of a dental implant that may be used in any of the first to fifth embodiments of the invention,

FIG. 20 is a schematic side sectional view of a dental implant identification system of the non-contact type showing only a dental implant and a non-contact tip of a reader device electrically connected to a match circuit, the configuration being according to a sixth embodiment of the invention,

FIG. 21 is a schematic side view of a radio frequency identification tag of a dental implant that may be used in the dental implant identification system of the non-contact type shown in FIG. 20,

FIG. 22 is a schematic end view of the radio frequency identification tag shown in FIG. 21,

FIG. 23 is a perspective view of a reader device that may be used in a dental implant identification system of either the non-contact type or the contact type,

FIG. 24 is a schematic side sectional view of a dental implant identification system of the contact type showing only a dental implant and a contact tip of a reader device electrically connected to a match circuit, the configuration being according to a seventh embodiment of the invention,

FIG. 25 is a schematic side view of a radio frequency identification tag of a dental implant that may be used in the dental implant identification system of the contact type shown in FIG. 24,

FIG. 26 is a block and circuit diagram showing the key electronic components and circuitry design of a reader device that can be used in the sixth embodiment of the invention, and showing its interaction with the radio frequency identification tag of the dental implant and with the information processing means, and

FIG. 27 is a block and circuit diagram showing the key electronic components and circuitry design of a reader device that can be used in the seventh embodiment of the invention, and showing its interaction with the radio frequency identification tag of the dental implant and with the information processing means.

### MODES FOR CARRYING OUT THE INVENTION

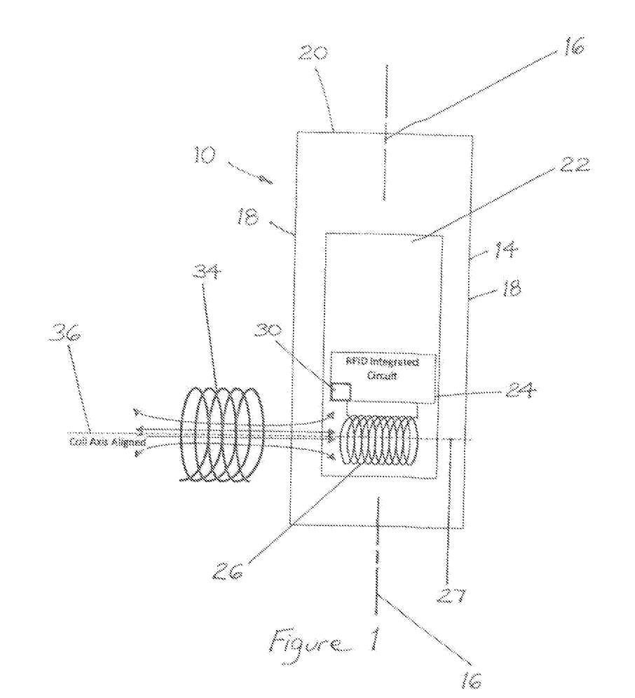

The dental implant identification system of the non-contact type shown schematically in FIG. 1 has a dental implant 10 and a single reader antenna coil 34 of a portable reader device, both of these components being in a first configuration for use of the system of the invention.

The dental implant 10, which is adapted for engagement to a jaw bone, has a generally cylindrical main body 14 or shank that is typically formed of a medical grade metal, such as titanium. The body 14 has a longitudinal axis 16 and an outer threaded surface 18 which is used for screwing the body 14 into the jaw bone in the direction of the longitudinal axis 16. The body 14 has an upper driving feature, such as a recess or socket, configured to receive a driving tool used to screwably engage the body into the bone.

An abutment or head portion extends from an upper axial end 20 of the body and is used for connecting a prosthetic tooth or crown thereto. There is a sealed or closed cavity 22 within the body 14, and positioned immovably inside the cavity 22 is a radio frequency identification (RFID) tag 24 or transponder which stores information for identification of the dental implant 10. The RFID tag may alternatively be embedded within a non-hollow body, such as by a process which moulds a non-metallic body around the tag.

The RFID tag is able to withstand gamma radiation sterilisation, autoclave sterilisation and other conditions it may encounter in normal use, both before and after oral installation, and must be capable of operating in or adjacent to metal objects.

The RFID tag 24 includes a receiver transmitter element which, in this embodiment suited to low frequency RFID systems, is in the form of a magnetic induction coil, referred to hereinafter as a tag antenna coil 26. In high frequency applications, the receiver transmitter element is in the form of a dipole antenna.

The tag antenna coil 26, which is typically a small wound ferrite coil, has a tag coil axis 27 which is orientated perpendicularly to the longitudinal axis 16 of the body 14 so that the axis 27 passes through the thinnest part (side walls) of the body 14 (as shown in FIG. 1) and is perpendicular to the jaw line. The orientation of the tag antenna coil 26 perpendicular to the longitudinal axis 16 of the body 14 will generally reduce the available size of the coil 26 since the coil's axis 27 is no longer parallel to the body's axis 16. However, this unusual orientation is important in that it provides the shortest distance to the reader's coil 34 and the radio frequency signal emanating from the tag antenna coil 26 passes through the smallest amount of metal of the body 14. Such factors will optimise performance and compensate for the smaller size of the tag antenna coil 26.

The importance of the tag coil axis 27 being orientated perpendicularly to the jaw line will also become apparent later in this specification. The upper driving feature of the body may be used to rotate the body until such an orientation is achieved, with the changing orientation being monitored by a visual indicator on the driving tool or by preliminary use of the reader device of the system to position the reader antenna coil at the desired perpendicular location and then monitor the strength of the radio frequency signal it receives from the tag antenna coil as the body is being rotated until a maximum signal strength is received.

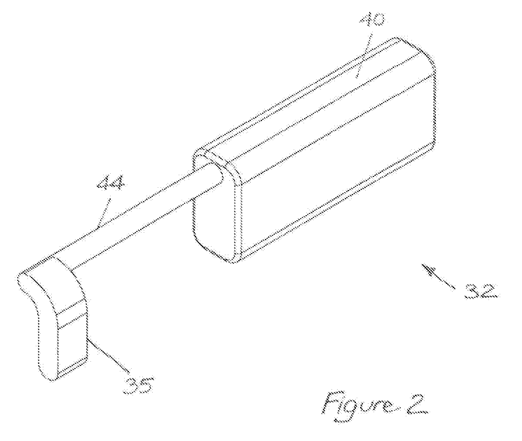

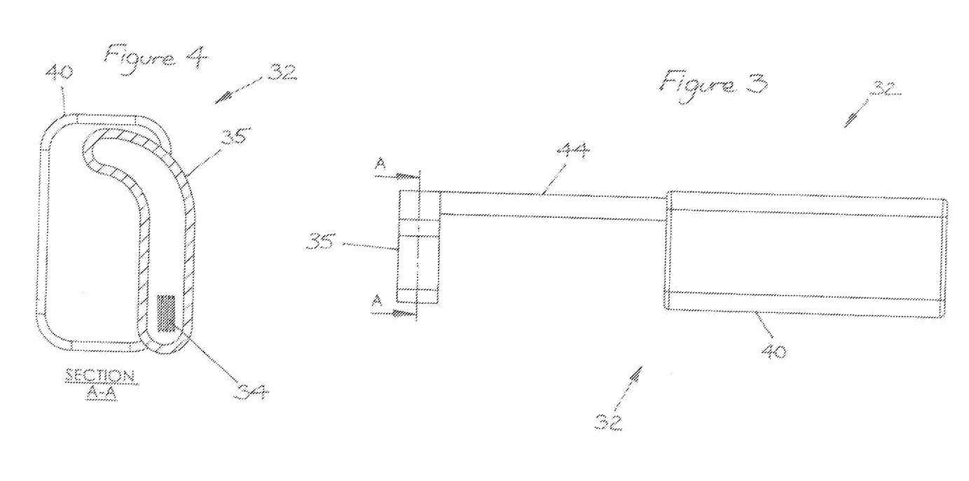

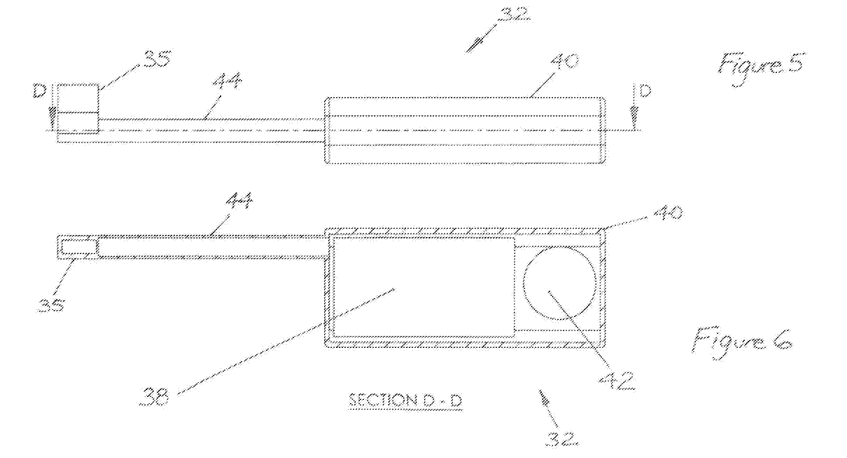

The reader antenna coil 34 is a component of the portable reader device 32 or scanner (shown in FIGS. 2 to 6) which can read or decode (or interrogate) the information stored in the RFID tag 24.

The reader device 32 is of the inductively magnetic field coupled type which use coil antennae and are effective over short distances. By utilising the passive RFID concept, the reader device 32 can provide a non-invasive method for the identification of information relating to the dental implant.

The reader device 32 includes a transmitter receiver element which, in this embodiment, is in the form of a power coil, referred to hereinafter as a reader antenna coil 34. In high frequency applications, the transmitter receiver element is in the form of a dipole transmitter. The reader antenna coil 34 is housed in an arcuate or generally J-shaped probe compartment 35 of the reader device 32. The arcuate shape of the probe compartment is to complement the shape of the jaw so that the probe compartment can “wrap around” the crown and its interconnected dental implant, thus bringing the reader antenna coil 34 as close as possible alongside, and in axial alignment with, the tag antenna coil positioned inside the body 14 of the dental implant.

The reader antenna coil 34 is connected to a conversion circuit which includes an oscillator that energises the coil 34 and an analogue-to-digital converter that converts variations in the current passing along the coil 34 to digital signals.

The coil 34 has a reader coil axis 36 which, in use, is ideally aligned with the tag coil axis 27 (as shown in FIG. 1). The coil 34 transmits signals to, and receives signals from, an integrated circuit mounted on an electronics board 38 housed in a handle 40 of the reader device 32. A rechargeable battery 42 for powering the reader device is also housed in the handle 40. The components in the handle 40 are operably connected to the components in the probe compartment 35 through an extension arm 44.

The RFID tag 24 is passively powered by electromagnetic wave transmissions from the reader antenna coil 34 of the reader device 32 and received by the tag antenna coil 26. In this way, the RFID tag 24 does not require an internal power supply that can be exhausted over time.

The information storage element 30 of the RFID tag 24 is an integrated circuit that is configured to generate a digital signal corresponding to the information that is stored in encoded form in the element 30. The received strength of this digital signal is maximised when the reader coil axis 36 is aligned with the tag coil axis 27, and this is more readily achieved due to the perpendicular orientation of the tag coil axis 27 to the jaw line, which means that the part of the reader device housing the reader antenna coil can be positioned similarly perpendicularly to the jaw line, where there is the most available space.

Upon activation of the RFID tag 24 by the reader device 32, this digital signal is fed back through the control circuit 28 which varies the resistance in the tag antenna coil 26 to transmit the encoded information stored in the element 30 as a digital signal to the reader antenna coil 34. The integrated circuit in the reader device 32 is correspondingly configured to translate the digital signal to a human readable format.

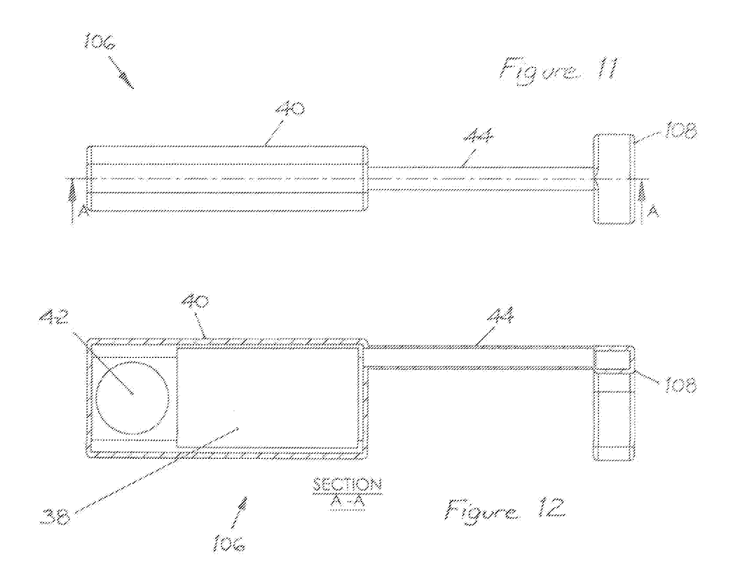

The key electronic components and circuitry design of a reader device 50 that can be used in various embodiments of the invention, and its interactions with the RFID tag 24 of the dental implant 10 and with a desk top computer 62 and server 64 of the information processing means, are shown in FIG. 13. Reader device 50 has a uniquely designed transceiver extension/interface circuit 52 which enables high performance electromagnetic field operation for extremely small sized RFID tags, such as tag 24. The extension/interface circuit 52 includes a transceiver circuit 34 that has two reader antenna coils 55, 56 and a resonant capacitor 57.

An alternate block and circuit diagram showing key electronic components and circuitry design of a reader device that is similar to that in FIG. 13 is shown in FIG. 14.

In more general terms, the reader device 32 transmits electromagnetic waves and the RFID tag in the dental implant is tuned to receive those waves through electromagnetic induction when the tag antenna coil and the reader antenna coil are magnetic field coupled. The RFID tag draws power from the field created by the reader device and uses it to power the circuits of its microchip. The microchip then modulates the waves that the RFID tag transmits back to the reader device, which converts the received waves into digital data. The digital data, as received, contains identification information on the particular dental implant being interrogated. This information may then be communicated wirelessly or by USB connection to information processing means, such as a computer, for processing the information into a human readable format.

The information cannot only relate to the dental implant itself, such as information as to the manufacturer , part number, batch number, and manufacture date, or any other unique identifier of the dental implant, all of which occur before the information storage element 30 is incorporated into the dental implant, but it can include patient information, date of oral installation, inspection dates and other information about events that have occurred in the life of the dental implant, both prior to and after its engagement to a taw bone.

In this case, remote writing features can be provided to the information storage element 30 that allow writing to a dental implant that has an already incorporated information storage element or has been orally installed, such as by radio frequency transmission. To achieve this, the control circuit 28 would be modified to permit switching the information storage element between “read” and “write” modes. The desired information can be written to the dental implant from the RFID tag 24 or from the information processing means.

The information processing means can be a server or host computer associated with the reader device and operable by the clinician, and which can access a database containing information relating to the identification information. Such a database can be stored locally on the host computer or can be accessed via a local area network or via the Internet as a centralised database. The database could be provided as a “Cloud” service. After the decoded RFID tag information is sent from the reader device to the computer, either wirelessly or via a USB connection, the computer compares that information with the information contained on the database and provides useful information to the clinician or other user of the system, such as it the information is identical. Information arising from this interrogation can be stored on the reader device and/or uploaded on the computer and/or added to the database.

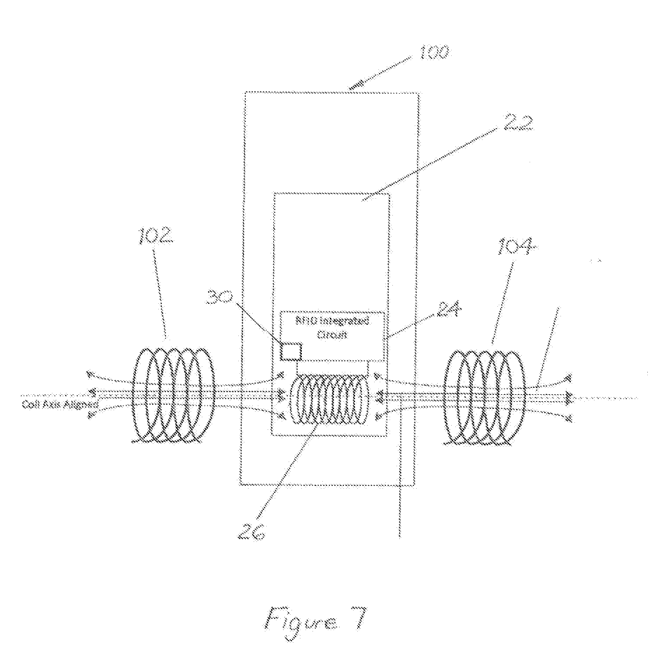

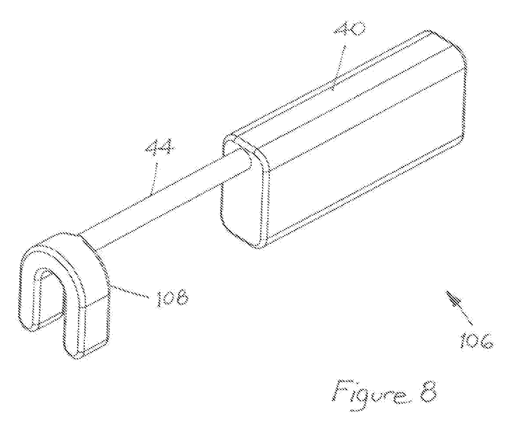

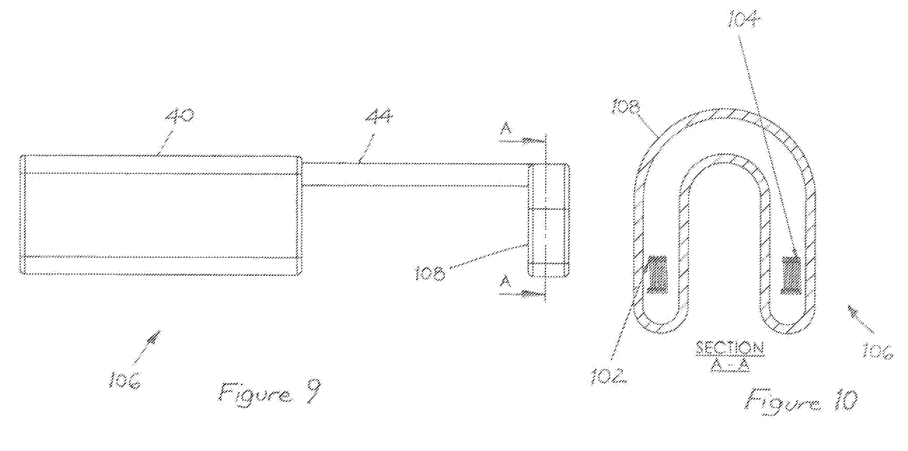

The dental implant identification system of the non-contact type shown schematically in FIG. 7 has a dental implant 100 and two reader antenna coils 102, 104 of a portable reader device 106 (shown in FIGS. 8 to 12), the coils 102, 104 being positioned at respective opposed sides of the dental implant 100 so as to have their axes aligned with. the axis of a tag antenna coil 26 positioned inside the dental implant 100, all of these components being in a second configuration for use of the system of the invention.

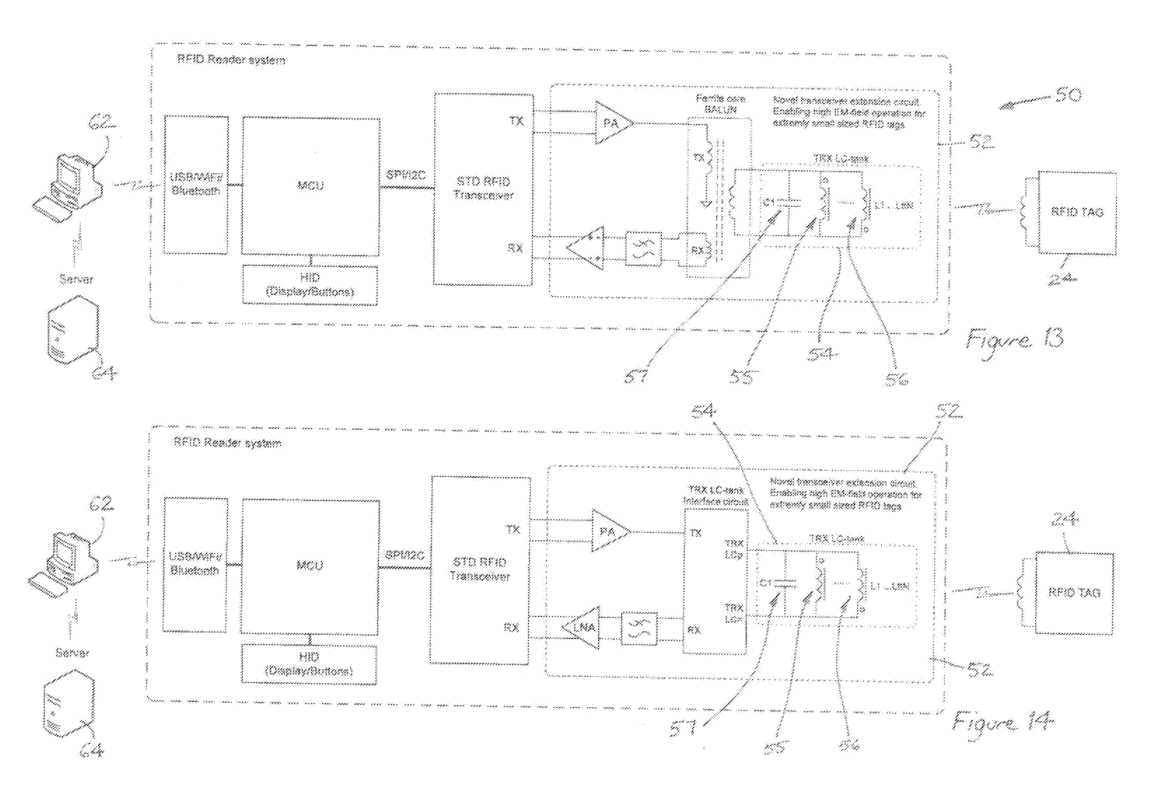

The main structural differences between this system as shown in FIGS. 7 to 12 and the system as shown in FIGS. 1 to 6 are that this system employs two reader antenna coils 102, 104 (not one such coil 34), and those coils 102, 104 are housed accordingly in a generally U-shaped probe compartment 108 (not a generally J-shaped probe compartment 35) of the reader device 106. There are other, more minor, structural differences which (along with their minor functional consequences) would be readily apparent to the skilled addressee of this specification and which are a consequence of the main structural differences indicated above. However, all other structural features (and their functional consequences) of the system as shown in, and described with reference to, FIGS. 1 to 6 are present in the system as shown in FIGS. 7 to 12, and so those other structural features (and their functional consequences) need not be again described herein with reference to FIGS. 7 to 12 but they are shown in FIGS. 7 to 12 with the same numerals used to identify those features in FIGS. 1 to 6. The skilled addressee of this specification would readily understand from the description and drawings of the structural features (and their functional consequences) of the system as shown in FIGS. 1 to 6 that the structural differences, both main and more minor, that are present or would be readily apparent to the skilled addressee, would not substantially affect the working operation or use of the system as shown in FIGS. 7 to 12 as compared to the working operation or use of the system as shown in, and described with reference to, FIGS. 1 to 6, but may affect the performance outcome of the system as shown in FIGS. 7 to 12 as compared to that in FIGS. 1 to 6. For example, the strength of the signal received by the reader device 106 employing the two reader antenna coils 102, 104 to transmit matching in-phase signals to the tag antenna coil 26 is effectively doubled as compared to the received signal strength of the reader device 32 employing one reader antenna coil 34. The same considerations relating to structural differences, working operation or use, and performance outcomes apply to the systems shown schematically in FIGS. 15, 17 and 18.

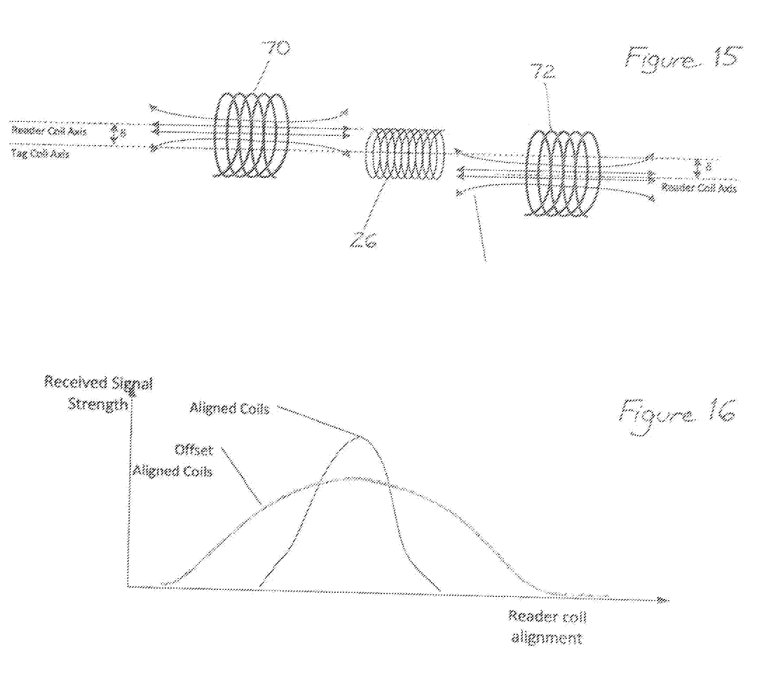

The dental implant identification system of the non-contact type shown schematically in FIG. 15 is the same as that shown in FIGS. 7 to 12 except that its two reader antenna coils 70, 72 have respective axes which, whilst they are parallel to the axis of the tag antenna coil 26, are deliberately offset from the tag antenna coil axis. This third configuration for use of the system of the invention is based on the fact that, in practice, precise alignment of the reader antenna coil axis with the tag antenna coil axis will be difficult to achieve. To broaden the effective response and improve robustness to misalignment the coil axes of a system employing multiple reader antenna coils can be deliberately misaligned. When, as shown in FIG. 15, the axes of two reader antenna coils are misaligned, both with each other and with the axis of the tag antenna coil, by an offset (δ), this will reduce the maximum (achievable) received signal strength (and so weaken the best case performance outcome) but increase the range of misalignment for which a useful and sufficiently strong signal Will be received by the reader antenna coil shown graphically in FIG. 16). Therefore, the system of FIG. 15 trades off peak performance with improved performance under misalignment.

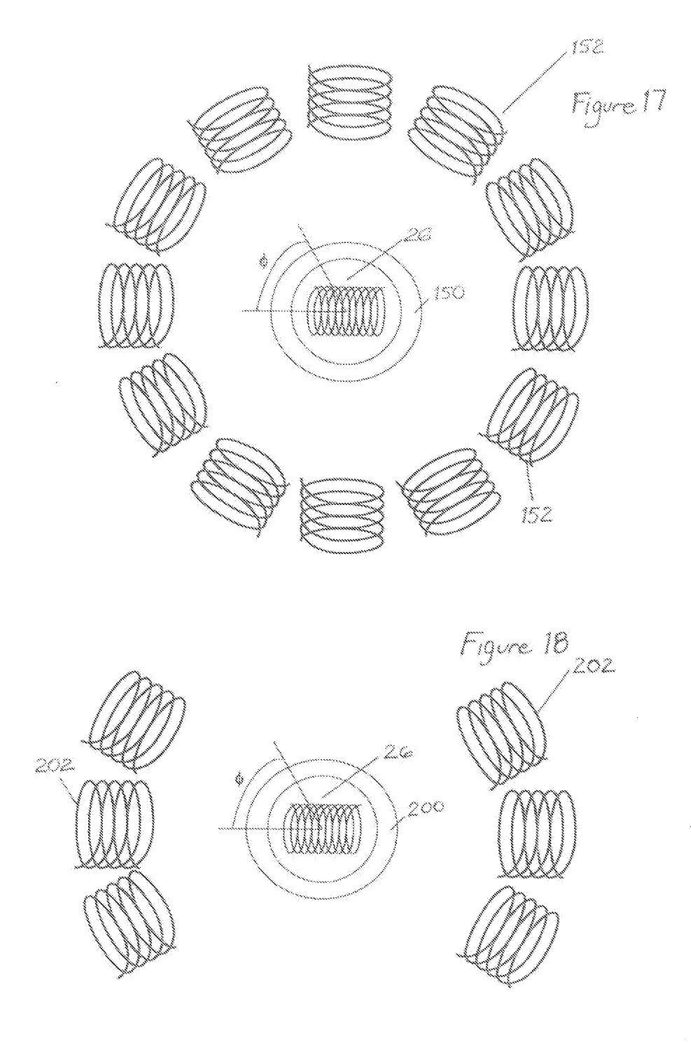

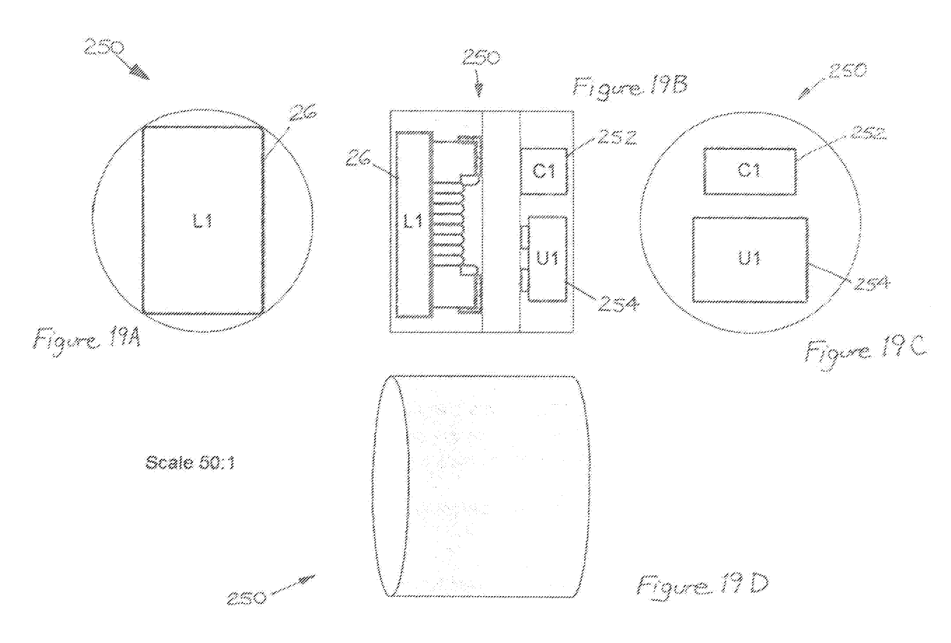

The dental implant identification system of the non-contact type shown schematically in FIG. 17 has a dental implant 150 and twelve reader antenna coils 152 of a portable reader device, the coils 152 being positioned at respective evenly spaced apart locations radially around the side of the dental implant so that at least two of those coils have their axes aligned substantially with the axis of the tag antenna coil 26 positioned inside the dental implant 150, all of these components being in a fourth configuration for use of the system of the invention.

The use of an increased number of twelve reader antenna coils in this arrangement is to further mitigate the effect of rotational misalignment. Any rotation& misalignment will be mitigated since additional sets of coils will become aligned axially with the axis of the tag antenna coil. This configuration may have limitations in some applications where this large number of coils and their location in the reader device may prevent the reader antenna coils being brought close enough to the RFID tag inside a dental implant, because of obstruction by objects surrounding the dental implant, to receive a useful and sufficiently strong signal.

In those applications where this problem exists, it may be overcome by reducing the number of reader antenna coils in the reader device and concentrating those remaining coils into two distinct and separated groups.

The dental implant identification system of the non-contact type shown schematically in FIG. 18 has a dental implant 200 and six reader antenna coils 202 of a portable reader device, the coils 202 being divided into two groups of three coils at respective opposed sides of the dental implant so that at least two of those coils have their axes aligned substantially with the axis of the tag antenna coil 26 positioned inside the dental implant 200, all of those components being in a fifth configuration for use of the system of the invention.

The use of a decreased number of six reader antenna coils in this arrangement is to overcome the problem of obstruction by objects surrounding the dental implant, although the system of FIG. 18 trades off peak performance to overcome this problem because of the now more limited degree of rotation of the reader device for which a useful and sufficiently strong signal will be received.

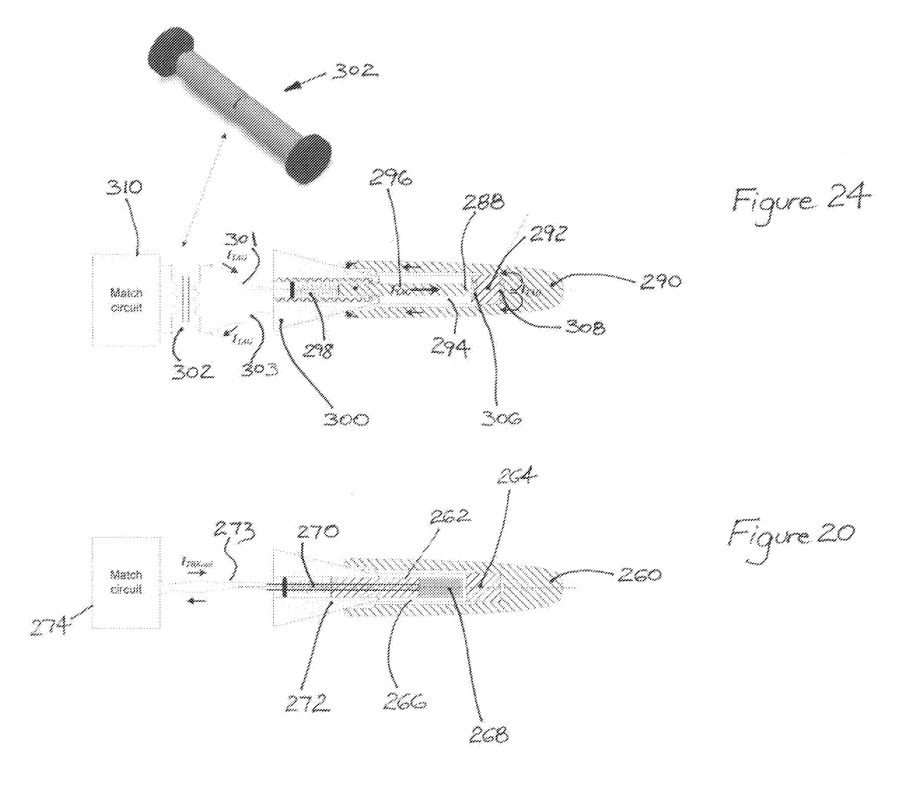

The double sided circuit board 250 shown in FIGS. 19A, 19B, 19C and 19B is used in the RFID tag 24 or any other RFID tag of a dental implant described above. The key electronic components are solder mounted on opposite surfaces of the circuit board 250. On one surface (see FIG. 19A) of the circuit board 250 is a receiver coil (L1) (or tag antenna coil) 26. On the other opposite surface (see FIG. 19C) of the circuit board 250 is a resonant capacitor (C1) 252 and an RFID integrated circuit (U1) 254.

The circuit board 250 is encapsulated as shown in FIG. 19D and the circuit board routes signals between the key electronic components 26, 252 and 254.

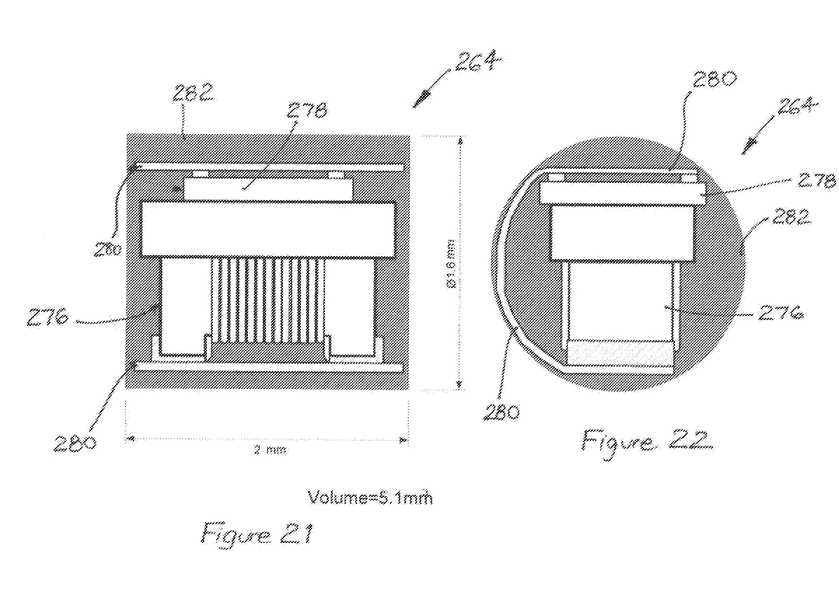

The dental implant identification system of the non-contact type shown schematically in FIG. 20 has a dental implant 260 and a non-contact tip 262 of a reader probe 263 (shown in FIG. 23), both of these components being in a sixth configuration for use of the system of the invention.

The dental implant 260 has a non-contact RFID tag 264, which is cylindrical in shape, positioned immovably inside an open cylindrical cavity 266 within the main body of the dental implant 260.

The non-contact tip 262 of the reader probe 263 includes, at its leading end, a reader antenna coil 268 (which is a transmitter receiver element in the form of a power coil), a coil positioning spring 270 and a reader positioning collar 272. The reader antenna coil 268 is electrically connected to, and receives its power through, wiring 273 from a match circuit 274 along which current flows (as shown by the direction of the arrows in FIG. 20).

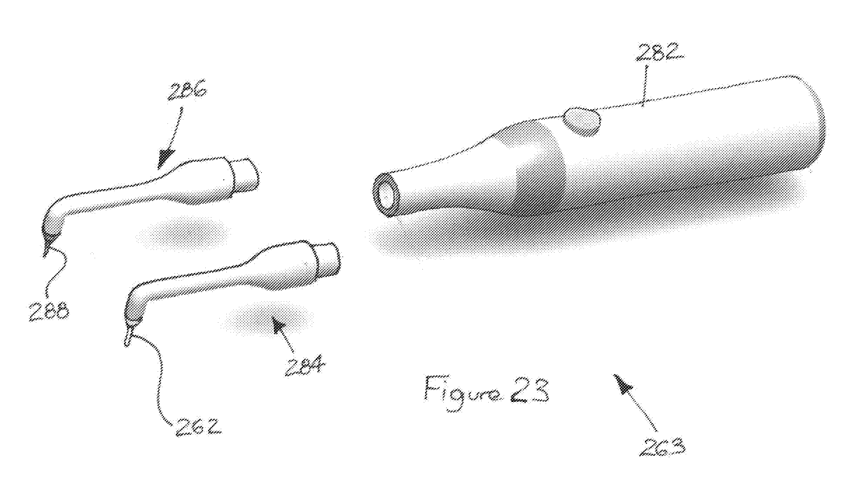

The non-contact RFID tag 264 (as shown in more detail in FIGS. 21 and 22) includes a surface mounted coil inductor, hereinafter referred to as a tag antenna coil 276, and an integrated circuit 278, both of which are mounted on a printed circuit board 280 and covered by a protective plastic moulding 282 which has high electrical resistance.

In use, the contact tip 262 of the “powered on” reader probe 263 is inserted through the opening of the cavity 266 of the dental implant 260 until further insertion is prevented by the reader positioning collar 272 becoming wedged in the opening, and the tag antenna coil 276 and the reader antenna coil 268 are magnetic field coupled. The non-contact RFID tag 264 is passively powered by electromagnetic wave transmissions from the reader antenna coil 268 of the reader probe and received by the tag antenna coil 276.

As shown in FIG. 23, the reader probe 263 has a handle 282 to which is connected a detachable nozzle 284 from which the non-contact tip 262 extends. As indicated above, the nozzle 284 is for use with the dental implant 260.

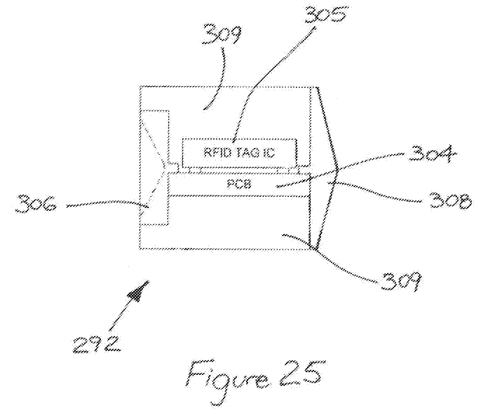

FIG. 23 also shows a detachable nozzle 286 which can alternatively be connected to the handle 282, and from which a contact tip 288 extends. The nozzle 286 is for use with a dental implant 290 as shown in, and to be hereinafter described with reference to, FIG. 24.

The dental implant identification system of the contact type shown schematically in FIG. 24 has a dental implant 290 and the contact tip 288 of the reader probe 263 (shown in FIG. 23), both of these components being in a seventh configuration for use of the system of the invention.

The dental implant 290 has a contact RFID tag 292 (shown in FIG. 25), which is cylindrical in shape, positioned immovably inside an open cylindrical cavity 294 within the main body of the dental implant 290.

The tip 288 of the reader probe 263 includes a reader contact terminal 296 at its leading end, a terminal pressure spring 298, and a reader positioning collar 300. The reader contact terminal 296 is electrically connected to and receives its power through, a current supply wiring 301 from a secondary side of an electrical transformer 302, and the reader positioning collar 300 is electrically connected in a current return wiring 303 to the transformer 302.

The contact RFID tag 292 includes a printed circuit board 304 on which is mounted an integrated circuit 305 between two contact electrodes 306, 308. The printed circuit board 304 and the integrated circuit 305 are covered by a protective plastic moulding 309 which has a high electrical resistance. The contact electrode 306 presents a contact surface in the shape of a conical depression facing towards the opening of the cylindrical cavity 294, and the contact electrode 308 presents a contact surface in the shape of a conical projection facing towards, and abutting, the base (or closed end) of the dental implant 290. Alternatively, the contact electrode 308 may present a contact surface in the shape of a ring facing outwardly and abutting a cylindrical wall region of the cavity at the base of the dental implant 290.

In use, the contact tip 288 of the “powered on” reader probe 263 is inserted through the opening of the cavity 294 of the dental implant 290, and the reader contact terminal 296 is pressed against, or contacts, the contact electrode 306 of the contact RFID tag 292 with low contact resistance and with a level of pressure regulated by the terminal pressure spring 298, until further insertion is prevented by the reader positioning collar 300 becoming wedged in the opening. The interconnection of the aforementioned components closes an electrical circuit along which current flows (as shown by the direction of the arrows in FIG. 24) between the transformer 302 of the reader probe 263 and the contact RFID tag 292. The transformer 302 is connected to a match circuit 310.

The handle 282 of the render probe 263 includes one or more buttons and a display, such as an LED display, connected to a microcontroller for facilitating the operation of the reader probe 263 by the user. The reader probe 263 is connected by wired or wireless means to a personal computer or other device operated through a microprocessor, such as a smart phone.

The reader probe 263 is “powered on” by a suitable power source which supplies power to standard operating components for RFID reader devices and either the match circuit 274 or the match circuit 310, depending on whether the dental implant identification system is of the non-contact type or of the contact type, housed within the reader probe 263.

Both the reader probe 263 of the portable reader device and either the non-contact RFID tag 264 or the contact RFID tag 292, operate preferably in the 13.56 MHz ISM band.

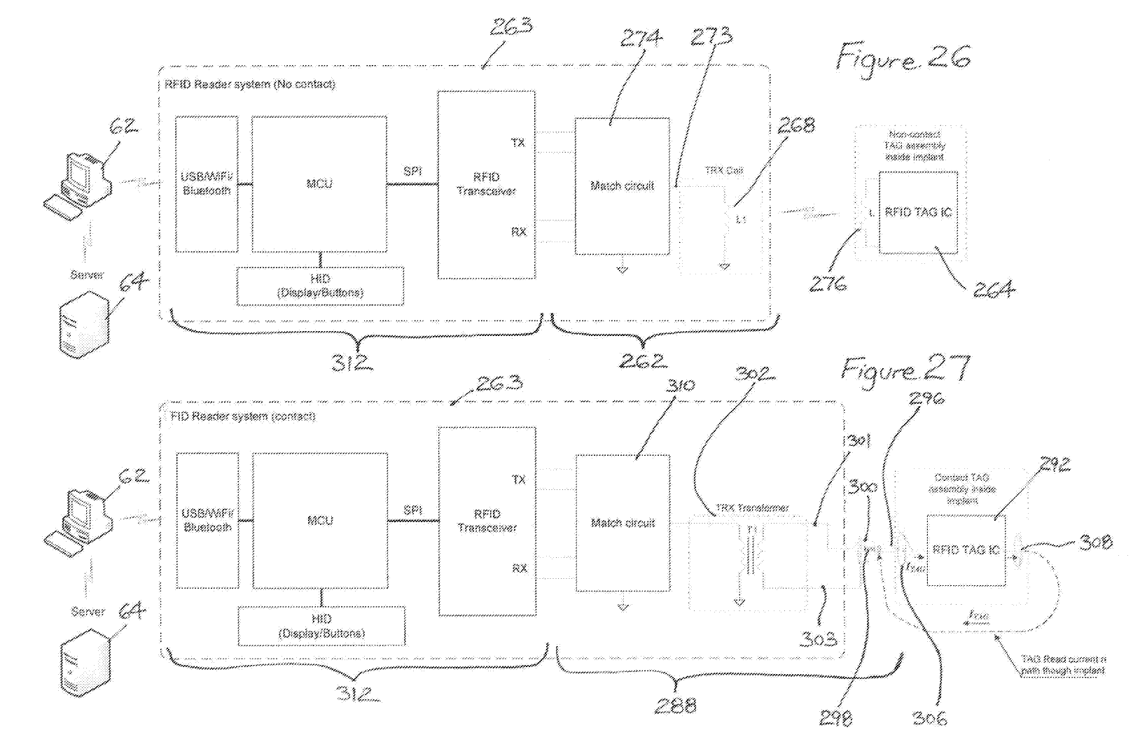

The main electronic components and circuitry design of the reader probe 263 which utilizes the non-contact tip 262, and its interactions with the RFID tag 264 of the dental implant 260 and with a desk top computer 62 and server 64 of the information processing means, are shown in FIG. 26. Reader probe 263 has a transceiver extension/interface circuit 112 which enables high performance electromagnetic field operation for extremely small sized RFID tags, such as tag 264, and also includes the match circuit 274 and the reader antenna coil 268 of the non-contact tip 262.

The main electronic components and circuitry design of the reader probe 263 which utilizes the contact tip 288, and its interactions with the RFID tag 292 of the dental implant 290 and with a desk top computer 62 and server 64 of the information processing means, are shown in FIG. 27. Reader probe 263 has a transceiver extension/interface circuit 312 which enables high performance electromagnetic held operation for extremely small sized RFID tags, such as tag 292, and also includes the match circuit 310, the transformer 302 and the reader contact terminal 296 of the contact tip 288.

The desk top computer 62 in both of the embodiments of FIGS. 26 and 27 could alternatively be a “laptop” or “note book” or other computer, or even a “tablet” computer or a smart phone.

As will be apparent to the skilled addressee of this specification, with the very large number of different types or brands of dental implants that have been made available, the dental implant identification system of the present invention provides an important aid to the dental professional by assisting them to identify which dental implant has been installed in a patient who requires restorative or other procedures on the implant. Not only can this information be used at the clinical level, it can also be used for inventory control, forensic identification and other types of investigation.

It will also be readily apparent to persons skilled in the art that various modifications may be made in details of design and construction of the embodiments of the dental implant identification system and devices, and in the steps of using the systems and devices described above, without departing from the scope or ambit of the present invention.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates before the filing date of this patent application.

1. A dental implant identification system of the non-contact type, comprising:
(a) a dental implant having a radio frequency identification tag which stores information for identification of the dental implant, the tag including a tag antenna coil having a first coil axis,
(b) a reader device for activating the tag and for reading the information stored on the tag when the information is transmitted from the tag upon, activation thereof, the reader device including a reader antenna coil having a second coil axis, and
(c) information processing means for processing the identification information read by the reader device,
wherein the radio frequency identification tag is so configured within the dental implant to provide an optimal reading of the identification information when the reader antenna coil is positioned alongside the tag antenna coil and the second coil axis is substantially aligned with the first coil axis, and when the reader device activates the tag.

2. The system of claim 1 wherein the radio frequency identification tag is located inside a cavity within a main body of the dental implant.

3. The system of claim 2 wherein the reader device is configured such that the reader antenna coil, when positioned alongside the tag antenna coil and when the first coil axis and the second coil axis are substantially aligned, is outside the main body of the dental implant.

4. The system of claim 2 wherein the reader device is configured such that the reader antenna coil, when positioned alongside the tag antenna coil and when the first coil axis and the second coil axis are substantially aligned, is inside the cavity of the main body of the dental implant.

5. The system of claim 1 wherein the information processing means is a computer associated with the reader device for accessing a database containing information relating to the identification information.

6. The system of claim 1 wherein the radio frequency identification tag further includes an information storage element and an integrated control circuit electrically connected between the tag antenna coil and the storage element and operable to activate the storage element in response to a voltage being generated in the tag antenna coil when the tag antenna coil and the reader antenna coil are magnetic field coupled.

7. The system of claim 1 wherein the identification information is stored on a microchip.

8. The system of claim 1 wherein the tag antenna coil operates as a passive power supply for the tag.

9. The system of claim 1 wherein the reader device has a single reader antenna coil.

10. The system of claim 1 wherein the reader device has two reader antenna coils.

11. A dental implant for engagement to a jaw hone, the dental implant comprising a main body having a longitudinal axis and an outer threaded surface for screwing the main body into the jaw bone in the direction of the longitudinal axis, the main both having an abutment for connecting a prosthetic tooth thereto, a cavity within the main body, a radio frequency identification tag positioned within the cavity for storing information for identification of the implant, the tag including an integrated circuit and a tag antenna coil having a tag coil axis that is orientated perpendicularly to the longitudinal axis of the main body, whereby the tag antenna coil of the radio frequency identification tag is able to be magnetic field coupled with a reader antenna coil of a reader device when the reader antenna coil is positioned alongside the tag antenna coil and when a reader coil axis of the reader antenna coil is orientated substantially perpendicularly to the longitudinal axis of the main body, the magnetic field coupling resulting in a voltage being generated in the tag antenna coil that is of a signal strength sufficient to cause the integrated circuit within the tag to be powered up so that the information can be read by the reader device.

12. A dental implant identification system of the contact type, comprising:
(a) a dental implant having a main body, a cavity Within the main body, and a radio frequency identification tag located inside the cavity, the radio frequency identification tag storing information for identification of the dental implant and including an integrated circuit between two contact electrodes,
(b) a reader device for activating the tag and for reading the information stored on the tag when the information is transmitted from the tag upon activation thereof, the reader device including a reader contact terminal, and
(c) information processing means for processing the identification information read by the reader device,
wherein the radio frequency identification tag is so configured within the dental implant to provide an optimal reading of the identification information when the reader contact terminal is inserted into the cavity and contacted against one of the contact electrodes, and when the reader device activates the tag.

13. The system of claim 12 wherein the information processing means is a computer associated with the reader device for accessing a database containing information relating to the identification information.

14. The system of claim 12 wherein the identification information is stored on a microchip.

15. A dental implant for engagement to a jaw bone, the dental implant comprising a main body having a longitudinal axis and an outer threaded surface for screwing the main body into the jaw bone in the direction of the longitudinal axis, the main body having an abutment for connecting a prosthetic tooth thereto, a cavity within the main body, a radio frequency identification tag positioned within the cavity for storing information for identification of the implant, the tag including an integrated circuit between two contact electrodes, whereby one of the contact electrodes of the radio frequency identification tag is able to be contacted by a reader contact terminal of a reader device the reader contact terminal is inserted within the cavity, the contact resulting in a voltage being generated in the in circuit that is of a signal strength sufficient to power up the integrated circuit so that the information can be read by the reader device.

16. The dental implant of claim 15 wherein the radio frequency identification tag includes a printed circuit board on which is mounted an, integrated circuit between a first contact electrode and a second contact electrode.

17. The dental implant of claim 16 wherein the printed circuit board and the integrated circuit are covered by a protective moulding that has a high electrical resistance.

18. The dental implant of claim 16 wherein the lust contact electrode presents a contact surface facing towards an opening of the cavity, and the second contact electrode presents a contact surface facing towards, and abutting a base of the dental implant.

19. The dental implant of claim 16 wherein, when a contact tip of a powered on reader device is inserted through an opening of the cavity in the main body and the reader contact terminal of the reader device is pressed against the first contact electrode of the radio frequency identification tag, and a reader positioning collar of the reader device is wedged simultaneously against the opening of the cavity in the main body, the simultaneous pressing and wedging closes an electrical circuit along which current flows between the reader device and the radio frequency identification tag of the dental implant.
